(19) United States
(12) Patent Application Publication
(10) Pub. No.: US 2021/0346252 A1
(43) Pub. Date: Nov. 11, 2021

(54) Remineralizing Oral Care Compositions Comprising Tin

(71) Applicant: The Procter & Gamble Company, Cincinnati, OH (US)

(72) Inventors: Arif Ali Baig, Mason, OH (US); Aaron Reed Biesbrock, Maineville, OH (US); Andrew Frederic Groth, Mason, OH (US); Samuel James St. John, Cincinnati, OH (US)

(21) Appl. No.: 17/308,085

(22) Filed: May 5, 2021

Related U.S. Application Data
(60) Provisional application No. 63/020,037, filed on May 5, 2020.

(51) Int. Cl. A61K 8/19; A61K 8/21; A61K 8/365; A61K 8/24; A61Q 11/00

(52) U.S. Cl. CPC A61K 8/19; A61K 8/21; A61K 8/365; A61K 2800/30; A61Q 11/00; A61K 2800/28; A61K 2800/51; A61K 8/24

(57) ABSTRACT

Dentifrice compositions including tin, monodentate ligand, polydentate ligand with low RDA values. Dentifrice compositions with a unique ratio of monodentate ligand to polydentate ligand to stabilize tin. Oral care compositions including tin with unexpectedly low stain at lower RDA values. Dentifrice compositions including tin and silica with an unexpectedly low stain and RDA value.

### FIELD OF THE INVENTION

The present invention is directed to oral care compositions comprising tin with unexpectedly low stain at lower RDA values. The present invention is also directed to dentifrice compositions comprising tin and silica with an unexpectedly low stain and RDA value.

### BACKGROUND OF THE INVENTION

Oral care compositions have included antimicrobial agents, such as tin ions, to counter oral bacteria and to prevent and treat conditions caused by bacteria in the oral cavity, such as formation of dental plaque, malodor and gum diseases. The formation of dental plaque and failure to stop their proliferation are the primary cause of dental caries, gingivitis, periodontal disease, and tooth loss. Additionally, tin ions can deposit on surfaces in the oral cavity to provide protective functions, such as antierosion, antibacterial, and/or antisensitivity benefits.

Calculus and plaque along with behavioral and environmental factors lead to formation of dental stains, significantly affecting the aesthetic appearance of teeth. Behavioral and environmental factors that contribute to teeth staining propensity include regular use of products that contain staining chemicals or color bodies such as coffee, tea, cola or tobacco and use of stain promoting oral products, such as those containing cationic antimicrobial agents.

Among the most common of cationic antimicrobial agents known to cause tooth staining are quaternary ammonium compounds such as cetylpyridinium chloride and metal ion sources such as stannous fluoride and stannous chloride. The tooth staining potential of these cationic materials has long been documented. Among the many approaches that have been suggested to reduce and control tooth staining and to whiten teeth is by the use of bleaches or oxidants such as peroxide. Essentially, bleaches act by oxidizing color bodies and existing stains. However, bleaches added to oral care products are typically present in low concentrations due to stability and safety limits. At these low concentrations, bleaches such as peroxide, are generally ineffective to control stain and whiten teeth. Furthermore, bleaches do not functionally act to prevent acquisition of stains.

Use of anionic ligands, particularly polymeric ones, have also been used to mitigate propensity of cation induced staining. However, stability and bioavailability of cationic antimicrobial ingredients are often compromised when formulated together with anionic stain mitigators. For example, polyphosphates are effective in mitigating CPC and stannous induced staining, but they affect the availability of both antimicrobials into dental plaque during use.

Another strategy to mitigate cationic antimicrobial agent staining is the use of highly abrasive materials, such as silica. Highly abrasive materials can remove these stains but can also remove layers of tooth surface if used in high amounts. Thus, there is a need for a dentifrice composition that includes tin, but does not require an abrasive with a high RDA value.

### SUMMARY OF THE INVENTION

Disclosed herein is a dentifrice composition comprising (a) tin, (b) abrasive, (c) monodentate ligand; (d) polydentate ligand; and (e) a pH of at least about 6, wherein the dentifrice composition has an RDA value of less than about 150.

Also disclosed herein is a dentifrice composition comprising (a) tin, (b) silica abrasive, (c) monodentate ligand comprising carboxylic acid; (d) polydentate ligand; and (e) a pH of at least about 6, wherein the dentifrice composition has an RDA value of less than about 150.

### BRIEF DESCRIPTION OF THE DRAWINGS

FIG. 1 shows the in vivo stain and RDA for Ex. 1-3.

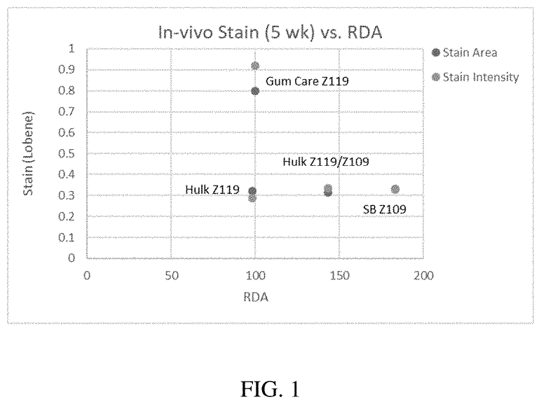

### DETAILED DESCRIPTION OF THE INVENTION

The present invention is directed to oral care compositions containing Sn that have an optimum stabilizing system for preventing the accumulation of Sn stain without the use of irritating chemical cleaning agents (e.g., polyphosphates) or high cleaning abrasive systems with (e.g., compositions with high RDA). The resulting invention provides efficacious oral hard tissue benefits without the accumulation of dental stain that is observed with poorly stabilized systems.

Poorly stabilized Sn is known to accumulate in dental plaque. As this unstabilized Sn is accumulated, it can convert from stannous ion to stannic ion, which can be dark yellowish brown shade. This dental stain can be difficult to remove and can require removal by a dental professional. Consumers upset by dental stain may choose a different toothpaste free of Sn and possibly missing important tooth and gum health benefits provided by bioavailable stannous ions. Traditionally, highly abrasive materials, such as particular silica parties are used to manage stain in toothpastes that have under-stabilized Sn or the accumulation of dental stain is possible.

Properly stabilizing stannous in oral care compositions to ensure efficacy while preventing stain formation requires the use of chelants. The chelate effect postulates that complexes of polydentate ligands with a metal are more stable than the dentate-normalized equivalent of the monodentate-ligand-stabilized metal complex (e.g., 1 mole of a bidentate ligand in comparison to 2 moles of a similarly structured monodentate ligand) because of a reduction in molar entropy of the bidentate chelate with respect to the monodentate complex.

While not wishing to be bound by theory, in the cases of metals forming complexes in mixed polydentate/monodentate solutions, configurational restrictions in bonding geometries often result when using conventional stabilizers (e.g., citrate anion) that thusly favor the formation of metal-monodentate-polydentate complexes. Consider the case of stannous metal ion being chelated by citrate anion. Sn²⁺ prefers a tetrahedral bonding geometry. The tridentate citrate anion can only occupy two of the four coordinating sites with stannous in this geometry because of steric restrictions. A monodentate ligand (e.g., gluconate) can thus participate in the complex at a third coordination site. The excess electron density (one electron from each of the three coordinating carboxylate anions minus the 2+ formal tin valency) is then distributed within the Sn bonding orbitals to the fourth coordination site that can acquire a hydrogen-bonded water or hydronium ion when in solution.

While not wishing to be bound by theory, if instead in the previous example, the molar ratio of citrate were increased from 1 to 2 and no monodentate ligand were present, the metal chelate would be over-stabilized resulting in a reduction of Sn bioavailability and a loss of oral care benefits. This is a direct result of the chelate effect. Additionally, the metal complex is under-stabilized if too little of the polydentate ligand is used in either the mixed- or polydentate-only cases also resulting in a loss of oral care benefits or the deposition of under-stabilized Sn leading to the accumulation of dental stain. Because of the unique geometric properties of stannous ion in solution (tetrahedral bonding geometry with 2+ formal valence) and in the presence of mixed mono/polydentate ligands, Sn²⁺ prefers mixed-dentate complexes. This is because, although two polydentate ligands can form a chelate complex, the resulting distribution of electron density is not favored thus providing an enthalpic penalty to formation of the complex.

Finally, in the case of monodentate-only stabilized metal complexes, there is no chelate effect and the stabilizing ligands can easily be replaced by chemical moieties with higher binding affinities. This results in under-stabilized stannous in the composition and loss to formula components (e.g., silica) over time or deposition of under stabilized Sn in the oral cavity causing the accumulation of dental stain. Unexpectedly, an optimum mixture of mono- and polydentate coordinating ligands is needed to properly stabilize to ensure efficacy and helping to prevent stain formation. As such, the present invention is directed to oral care compositions that contain Sn but provide exceptional stain control at a low RDA value.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

The term RDA refers to Relative Dentin Abrasion or Radioactive Dentin Abrasion as defined in FDI-ISO 11609.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.” All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The oral care composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, prophy paste or denture care or adhesive product. The components of the oral care composition can be incorporated into a film, a strip, a foam, or a fiber-based dentifrice composition.

The oral care compositions, as described herein, comprise tin, monodentate ligand, and polydentate ligand. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, for example, oxalic acid, or salts thereof, can be a dicarboxylic acid, a polydentate ligand, and/or a whitening agent.

### Tin

The oral care composition of the present invention comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. The tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source.

### Monodentate Ligand

The oral care composition comprises a monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The monodentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof.

The oral care composition can include from about 0.01% to about 10%, from about 0.1% to about 15%, from about 1% to about 5%, or from about 0.0001 to about 25%, by weight of the composition, of the monodentate ligand.

### Polydentate Ligand

The oral care composition comprises polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e. with two functional groups), tridentate (i.e. with three functional groups), tetradentate (i.e. with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

Suitable compounds comprising phosphate include orthophosphate, phosphate, polyphosphate, salts thereof, and/or combinations thereof. Suitable phosphate compounds include phosphate salts, organophosphates, or combinations thereof. Suitable phosphate salts include salts of orthophosphate, hydrogen phosphate, dihydrogen phosphate, alkylated phosphates, polyphosphates, and/or combinations thereof.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can comprise oxalic acid, oxalic acid, malonic acid, succinic acid, glutaric acid, adipic acid, pimelic acid, suberic acid, azerlaic acid, sebacic acid, undecanedioic acid, dodecanedioic acid, bras sylic acid, thapsic acid, japanic acid, phellogenic acid, equisetolic acid, malic acid, tartaric acid, citric acid, phytic acid, pyrophosphate, tripolyphosphate, tetrapolyphosphate, hexametaphoshate, salts thereof, and/or combinations thereof.

The oral care composition can include from about 0.01% to about 10%, from about 0.1% to about 15%, from about 1% to about 5%, or from about 0.0001 to about 25%, by weight of the composition, of the polydentate ligand.

### Ratio of Tin to Monodentate Ligand to Polydentate Ligand

The oral care composition, as described herein, comprises a ratio of tin to monodentate ligand to polydentate ligand that provides an unexpectedly high amount of soluble tin and/or a superior fluoride uptake. Suitable ratios of tin to monodentate ligand to polydentate ligand can be from about 1:0.5:0.5 to about 1:5:5, from about 1:0.5:0.75 to about 1:5:5, from about 1:1:1 to about 1:5:5, from about 1:1:0.5 to about 1:2.5:2.5, from about 1:1:1 to about 1:2:2, from about 1:0.5:0.5 to about 1:3:1, or from about 1:0.5:0.5 to about 1:1:3.

Desired herein are oral care compositions with a soluble Sn of at least about 1000 ppm, 2000 ppm, 4000 ppm, at least about 4500 ppm, at least about 5000 ppm, at least about 6000 ppm, and/or at least about 8000 ppm. Also desired herein are oral care compositions with a fluoride uptake of at least about 6.5 μg/cm², at least about 7.0 μg/cm², at least about 8.0 μg/cm², or at least about 9.0 μg/cm² after a time period of at least about 9 days, 30 days, 65 days, 75 days, 100 days, 200 days, 365 days and/or 400 days.

In total, while not wishing to be bound by theory it is believed that the soluble Sn amount is correlated to bioavailable Sn as it is freely available to provide an oral health benefit. Fully bound Sn (i.e. Sn that is overchelated) or precipitated Sn (i.e. insoluble tin salts, such as Sn(OH)2 and/or Sn-based stains can form when Sn is underchelated) would not be included in the measurement for soluble Sn. Additionally, while not wishing to be bound by theory, it is believed that a carefully balanced ratio of Sn to monodentate and polydentate ligands can provide a high amount of bioavailable fluoride and Sn ions without some of the negatives to the use of cationic antimicrobial agents, such as surface staining. Thus, additional screening experiments were done to quantify and qualify the ranges and identities of monodentate and polydentate ligands.

### Dicarboxylic Acid

The polydentate ligand can comprise dicarboxylic acid. The dicarboxylic acid comprises a compound with two carboxylic acid functional groups. The dicarboxylic acid can comprise a compound or salt thereof defined by Formula I.

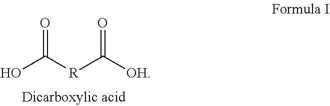

R can be null, alkyl, alkenyl, allyl, phenyl, benzyl, aliphatic, aromatic, polyethylene glycol, polymer, O, N, P, and/or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.01% to about 10%, from about 0.1% to about 15%, from about 1% to about 5%, or from about 0.0001 to about 25%, by weight of the oral care composition, of dicarboxylic acid.

### Tricarboxylic Acid

The polydentate ligand can comprise tricarboxylic acid. The tricarboxylic acid comprises a compound with three carboxylic acid functional groups. The tricarboxylic acid can comprise a compound or salt thereof defined by Formula II.

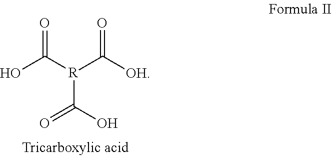

R can be alkyl, alkenyl, allyl, phenyl, benzyl, aliphatic, aromatic, polyethylene glycol, polymer, O, N, P, and/or combinations thereof.

The tricarboxylic acid can comprise citric acid, isocitric acid, aconitic acid, propane-1,2,3-tricarboxcylic acid, trimesic acid, any tricarboxylic acid in the citric acid cycle or Krebs Cycle, salts thereof, or combinations thereof. The tricarboxylic acid can comprise suitable salts of tricarboxylic acid, such as for example, sodium citrate.

The oral care composition can comprise from about 0.01% to about 10%, from about 0.1% to about 15%, from about 1% to about 5%, or from about 0.0001 to about 25%, by weight of the oral care composition, of tricarboxylic acid.

### Polyphosphate

The polydentate ligand can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is so potassium, ammonium, or any other alkali metal cations and n averages frons about 2 to about 21, from about 2 to about 14, or from about 2 to about 7. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate. The oral care composition can be essentially free of, substantially free of, or free of cyclic polyphosphate. The oral care composition can be essentially free of, substantially free of, or free of phytic acid.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.5% to about 1.5%, or from about 0.3% to about 0.6%, by weight of the dentifrice composition. In particular, zinc can be detrimental to the remineralization process. Thus, the oral care composition can be essentially free of, substantially free of, or free of zinc.

pH

The pH of the oral care compositions as described herein can be from about 4 to about 7.5, from about 4.5 to about 6.5, or from about 4.5 to about 5.5. The pH of the oral care compositions, as described herein, can also be at least about 6, at least about 6.5, or at least about 7. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water. The pH of the oral care compositions as described herein have a preferred pH of from about 4 to about 10, from about 5 to about 9, from about 6 to 8, or about 7.

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

### Amino Acid

The oral care composition can comprise amino acid. The monodentate and/or polydentate ligand can comprise amino acid. Whether the amino acid is a monodentate ligand or polydentate ligand can be based on how many functional groups capable of chelating to, associating with, and/or bonding to tin are present and/or the pH of the oral care composition. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Suitable amino acids include the compounds described by Formula III, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

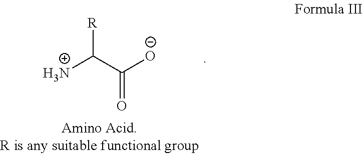

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acid which has an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the tin, monodentate ligand, and/or polydentate ligand include emulsion compositions—such as the emulsions compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Methods

The oral care compositions, as described herein, can lead to oral health benefits, such as the remineralization of teeth, when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure. The user can also be instructed to rinse with a mouthwash composition comprising a therapeutic amount of fluoride and/or mouth rinse composition comprising a therapeutic amount of fluoride after the completion of the brush procedure. The user can also be instructed to not rinse with any liquid, including tap or bottled water, other than a composition comprising a therapeutic amount of fluoride. As the application of the oral care composition can lead to oral health benefits, such as the remineralization of teeth, rinsing the oral cavity after application and expectoration of the oral care composition can remove residual fluoride from the surface of teeth, thereby at least partially diminishing the oral health benefit.

Other oral health benefits that can result from the use of the oral care composition in an oral cavity, such as in the application of the oral care composition to teeth, include increasing the density of teeth, the prevention of the loss of calcium from the teeth, repairing structural weaknesses in enamel, extending the life of a user's teeth, increasing the structural density of enamel, coating enamel with rebuilding minerals, and/or remineralization of teeth.

Disclosed herein are methods for increasing the density of teeth, the prevention of the loss of calcium from the teeth, repairing structural weaknesses in enamel, extending the life of a user's teeth, increasing the structural density of enamel, coating enamel with rebuilding minerals, and/or remineralization of teeth comprising instructing a user to apply an oral care composition, as described herein, for at least 1 minute twice a day. The method can also include instructing a user to expectorate the oral care composition and either not rinsing the oral cavity or only rinsing the oral cavity with a composition comprising a therapeutic amount of fluoride.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Human In Vivo Stain

The objective of this study was to evaluate the stain accumulation potential of two experimental dentifrices containing stannous fluoride relative to two reference formulas. The results presented here are from a randomized, parallel group, double-blind, single-center study with four treatment groups comprised of healthy adult volunteers. It was further preferred that volunteers present with accumulated natural stain at their baseline visit and to drink at least one cup of coffee per day, ore more preferably two cups of coffee per day. At the baseline visit, subjects received an oral exam and a Lobene stain exam according to the method published by Lobene et al. (JADA, 77:849-855, 1968), followed by a prophylactic cleaning on the twelve anterior teeth. Subjects were then randomly assigned to one of the four dentifrice treatments. Subjects were then given a study kit including a soft manual brush (Oral-B Indicator, Soft, Procter & Gamble, OH, USA), the appropriate toothpaste, a timer, and written instructions. The instruction sheet was verbally reviewed with the subjects who were asked to brush their teeth for one minute, twice daily (morning and evening) throughout the 5-week duration of the study. Safety and efficacy measurements were assessed at Baseline, Week 2, and Week 5.

The Lobene composite stain, stain area, and stain intensity scores at Week 5 were of primary interest; the Week 2 results were of secondary interest. Each pair of treatment groups were analyzed using analysis of covariance (ANCOVA). The ANCOVA model included treatment as a factor and baseline stain score as a covariate. In the event that the normality or equal variance assumption for parametric analysis was statistically significantly violated, data transformation and/or nonparametric methods were used. Statistical comparisons were two-sided with a significance level of 0.05.

### Relative Dentin Abrasion (RDA)

The Relative Dentin Abrasion (RDA) test is typically performed to confirm that a dentifrice composition, e.g., toothpaste, is safe for consumer use, with the recommended upper limit of the test set at 250. The RDA values in TABLE 2 and TABLE 3 were determined by using the industrial published standard as outlined in FDIS-ISO 11609, Annexure, third edition Annex B: Determination of relative dentifrice abrasivity to enamel and dentine by a surface profile method, which is herein incorporated by reference.

TABLE 1
Oral Care Compositions

| | Example 1 | Example 2 | Example 3 |
|---|---|---|---|
| Glycerin | 27.013 | 27.013 | |
| Sorbitol | 34.900 | 34.900 | 48.0000 |
| Water | 8.186 | 8.186 | 20.8311 |
| Stannous Fluoride | 0.454 | 0.454 | 0.4540 |
| Stannous Chloride (10% Silica) | 0.4400 | 0.4400 | 0.5619 |
| Zinc Citrate | — | — | 0.5330 |
| Sodium Gluconate | 1.020 | 1.020 | 1.3000 |
| Sodium citrate | 1.220 | 1.220 | — |
| Silica Z109 | — | 7.500 | 17.5000 |
| Silica Z119 | 15.000 | 7.500 | — |
| Carrageenan | 1.050 | 1.050 | 1.5000 |
| Xanthan Gum | 0.6125 | 0.6125 | 0.8750 |
| Sodium Lauryl Sulfate 28% | 5.625 | 5.625 | 5.0000 |
| Soln | | | |
| Betaine (30% Soln) | 1.500 | 1.500 | |
| Flavor | 1.100 | 1.100 | 1.1750 |
| NaOH 50% | 0.720 | 0.720 | 0.8700 |
| Saccharin | 0.500 | 0.500 | 0.4000 |
| Sucralose liquid | 0.160 | 0.160 | 0.2000 |
| Titanium Dioxide | 0.500 | 0.500 | 0.5000 |
| Dye | — | — | 0.3000 |

TABLE 2
Stain and RDA at 2 Weeks

| | | | | Intensity | | |
|---|---|---|---|---|---|---|
| Treatment | N | Area stain | Stdev | stain | Stdev | RDA |
| Crest ® Gum Care | 23 | 0.414 | 0.063 | 0.485 | 0.067 | 110 |
| Ex. 1 (Inventive) | 23 | 0.244 | 0.052 | 0.237 | 0.049 | 98 |
| Ex. 2 (Inventive) | 23 | 0.354 | 0.08 | 0.372 | 0.082 | 143 |
| Ex. 3 (Comparative) | 23 | 0.254 | 0.034 | 0.262 | 0.052 | 183 |

TABLE 3
Stain and RDA at 3 Weeks

| | | | | Intensity | | |
|---|---|---|---|---|---|---|
| Treatment | N | Area stain | Stdev | stain | Stdev | RDA |
| Crest ® Gum Care | 23 | 0.798 | 0.105 | 0.918 | 0.093 | 100 |
| Ex. 1 (Inventive) | 23 | 0.319 | 0.319 | 0.288 | 0.038 | 98 |
| Ex. 2 (Inventive) | 23 | 0.313 | 0.313 | 0.334 | 0.058 | 143 |
| Ex. 3 (Comparative) | 22 | 0.332 | 0.332 | 0.326 | 0.06 | 183 |

TABLE 1 shows Examples 1-3. Example 1 and 2 are inventive examples with a suitable tin to monodentate ligand to polydentate ligand molar ratio, such as greater than 1:0:5:0.5, as described herein. Example 3 is a comparative example that has a tin to monodentate ligand to polydentate ligand molar ratio of below 1:0:5:0.5.

Examples 1-3 were compared to Crest® Gum Care (CGC), a stannous fluoride-based toothpaste known to cause tooth staining (i.e. a positive control). The tin to monodentate ligand to polydentate ligand molar ratio of CGC is 1:1:0. Use of CGC (RDA of 110) for 2 weeks led to an average area of stain of 0.414 with an average stain intensity of 0.485. After 5 weeks, CGC (RDA of 100) had an average stain area of 0.798 and an average stain intensity of 0.918.

In contrast, Example 3, a composition with a much higher RDA of 183, had an average stain area of 0.252 and an average stain intensity of 0.262 after 2 weeks and an average stain area of 0.332 and an average stain intensity of 0.326 after 5 weeks. While this stain area and stain intensity are improved, it required a much higher RDA. Prolonged use of toothpastes with high RDA values (such as >200) can contribute to enamel and dental loss. Thus, it would be desirable for an oral care composition to have the RDA value of CGC, but the staining potential of Example 3.

As shown in TABLE 2 and TABLE 3, Example 1 and 2 provide superior staining performance to Example 3 with unexpectedly low RDA values. Example 1 (RDA of 98) had an average stain area of 0.244 and an average stain intensity of 0.237 after 2 weeks and an average stain area of 0.319 and average stain intensity of 0.288 after five weeks. Example 1 unexpectedly outperformed Example 3, but with a much lower RDA value.

Example 2 (RDA of 143) had an average stain area of 0.354 and an average stain intensity of 0.372 after 2 weeks and an average stain area of 0.313 and average stain intensity of 0.334 after five weeks. Example 2 unexpectedly performed similar to Example 3, but with a much lower RDA value.

While not wishing to be bound by theory, it is believed that this superior stain performance was feasible with lower RDA values because the tin ions were properly chelated, thereby maximizing the amount of soluble tin ions without contributing to staining.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice composition comprising:
(a) tin;
(b) abrasive;
(c) monodentate ligand;
(d) polydentate ligand; and
(e) a pH of at least about 6,
wherein the dentifrice composition has an RDA value of less than about 150.

2. The dentifrice composition of claim 1, wherein the dentifrice composition is essentially free of zinc.

3. The dentifrice composition of claim 2, wherein the dentifrice composition is substantially free of zinc.

4. The dentifrice composition of claim 1, wherein the tin comprises stannous fluoride, stannous chloride, or combinations thereof.

5. The dentifrice composition of claim 1, wherein the abrasive comprises silica abrasive, calcium abrasive, or combinations thereof.

6. The dentifrice composition of claim 5, wherein the calcium abrasive comprises calcium carbonate.

7. The dentifrice composition of claim 1, wherein the dentifrice composition has a tin to monodentate ligand to polydentate molar ratio of from about 1:0.5:0.5 to about 1:5:5.

8. The dentifrice composition of claim 7, wherein the molar ratio of tin to monodentate ligand to polydentate ligand is from about 1:1:1 to about 1:2.5:2.5.

9. The dentifrice composition of claim 1, wherein the dentifrice composition comprises fluoride.

10. The dentifrice composition of claim 9, wherein the fluoride comprises stannous fluoride, sodium fluoride, sodium monofluorophosphate, amine fluoride, or combinations thereof.

11. The dentifrice composition of claim 1, wherein the dentifrice composition comprises no added water.

12. The dentifrice composition of claim 1, wherein the dentifrice composition comprises water.

13. The dentifrice composition of claim 1, wherein the dentifrice composition comprises up to 45%, by weight of the composition, of water.

14. The dentifrice composition of claim 1, wherein the monodentate ligand comprises a compound comprising a single functional group capable of chelating tin.

15. The dentifrice composition of claim 14, wherein the monodentate ligand comprises carboxylic acid or a salt thereof.

16. The dentifrice composition of claim 15, wherein the carboxylic acid comprises aliphatic carboxylic acid, aromatic carboxylic acid, sugar acid, salts thereof, or combinations thereof.

17. The dentifrice composition of claim 16, wherein the sugar acid comprises aldonic acid, ulsonic acid, uronic acid, aldaric acid, salts thereof, or combinations thereof.

18. The dentifrice composition of claim 16, wherein the sugar acid comprises gluconate.

19. The dentifrice composition of claim 16, wherein the aliphatic carboxylic acid comprises linear saturated carboxylic acid, linear unsaturated carboxylic acid, alpha hydroxy acid, beta hydroxy acid, gamma hydroxy acid, amino acid, salts thereof, or combinations thereof.

20. The dentifrice composition of claim 19, wherein the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

21. The dentifrice composition of claim 19, wherein the alpha hydroxy acid comprises lactate.

22. The dentifrice composition of claim 1, wherein the polydentate ligand comprises a compound comprising at least two functional groups capable of chelating tin.

23. The dentifrice composition of claim 22, wherein the polydentate ligand comprises carboxylic acid, polyphosphate, salts thereof, or combinations thereof.

24. The dentifrice composition of claim 23, wherein the carboxylic acid comprises dicarboxylic acid, tricarboxylic acid, salts thereof, or combinations thereof.

25. The dentifrice composition of claim 24, wherein the dicarboxylic acid comprises a compound with the formula HO₂C—R—CO₂H.

26. The dentifrice composition of claim 25, wherein R is aliphatic, aromatic, or combinations thereof.

27. The dentifrice composition of claim 24, wherein the dicarboxylic acid comprises oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, salts thereof, or combinations thereof.

28. The dentifrice composition of claim 24, wherein the tricarboxylic acid comprises citric acid, isocictric acid, aconitic acid, propane-1,2,3-tricarboxylic acid, trimesic acid, salts thereof, or combinations thereof.

29. The dentifrice composition of claim 23, wherein the polyphosphate comprises pyrophosphate, tripolyphosphate, tetrapolyphosphate, hexametaphosphate, or combinations thereof.

30. The dentifrice composition of claim 1, wherein the RDA is less than about 125.
